(19) United States
(12) Patent Application Publication
(10) Pub. No.: US 2009/0203744 A1
(43) Pub. Date: Aug. 13, 2009

(54) NOVEL PYRIDINE DERIVATIVES

(76) Inventors: Surendrakumar Satyanarayan Pande y, Chennai (IN); Gaddam Om Reddy, Chennai (IN); Gajendra Singh, Chennai (IN); Santhanagopalan Chithra, Chennai (IN); Jeganath Sivakumar, Chennai (IN); Venkatachalapathi Sanjay Kadnur, Chennai (IN); Debendranath Dey, Union City, CA (US)

Correspondence Address: GREENBLUM & BERNSTEIN, P.L.C., 1950 ROLAND CLARKE PLACE, RESTON, VA, 20191, US

(21) Appl. No.: 11/997,064

(22) PCT Filed: Jul. 28, 2006
(86) PCT No.: PCT/IB06/02064

§ 371 Date: Jun. 13, 2008

(30) Foreign Application Priority Data
Jul. 29, 2005 (IN) 1038/CHE/2005

(51) Int. Cl. A61K 31/4439; C07D 413/12

(52) U.S. Cl. 514/340; 546/271.4

(57) ABSTRACT

The present invention relates to novel pyridine derivatives of the formula (I), their stereoisomers, and their pharmaceutically acceptable salts, and compositions. The present invention more particularly provides novel pyridine derivatives of the general formula (I).

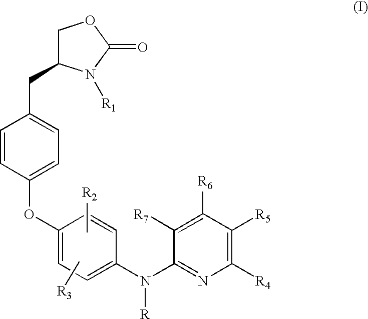

### FIELD OF THE INVENTION

The present invention relates to novel pyridine derivatives of the formula (I), their stereoisomers, and their pharmaceutically acceptable salts, and compositions. The present invention more particularly provides novel pyridine derivatives of the general formula (I)

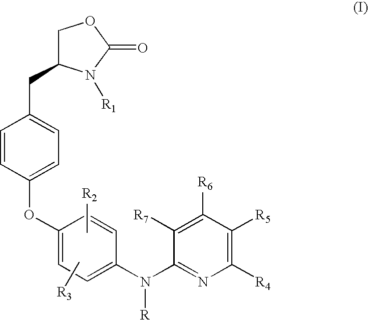

The present invention also relates to a process for the preparation of the above said novel compounds, their stereoisomers, and their pharmaceutically acceptable salts, and compositions. The compounds of the present invention are effective in lowering blood glucose, serum insulin, free fatty acids, cholesterol and triglyceride levels and are useful in the treatment and/or prophylaxis of type II diabetes. The compounds of the present invention are effective in treatment of obesity, inflammation, autoimmune diseases such as multiple sclerosis and rheumatoid arthritis. Surprisingly, these compounds increase the leptin level and have no liver toxicity.

Furthermore, the compounds of the present invention are useful for the treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1, IL-6, IL-1β and cyclooxygenase such as COX-2.

### BACKGROUND OF THE INVENTION

The causes of type I and II diabetes are not yet clear, although both genetics and environment seem to be the factors. Type I is an autonomic immune disease and the patient must take insulin to survive. Type II diabetes is a more common form and is a metabolic disorder resulting from the body's inability to make a sufficient amount of insulin or to properly use the insulin that is produced. Insulin secretion and insulin resistance are considered the major defects, however, the precise genetic factors involved in the mechanism remain unknown.

Patients with diabetes usually have one or more of the following defects:

Less production of insulin by the pancreas;

Over secretion of glucose by the liver;

Independence of the glucose uptake by the skeletal muscles;

Defects in glucose transporters, desensitization of insulin receptors; and

Defects in the metabolic breakdown of polysaccharides.

Other than the parenteral or subcutaneous administration of insulin, there are about four classes of oral hypoglycemic agents used i.e. sulfonylurea, biguanides, alpha glucosidase inhibitors and thiazolidinediones.

Each of the current agents available for use in the treatment of diabetes has certain disadvantages. Accordingly, there is a continuing interest in the identification and development of new agents, which can be orally administered, for use in the treatment of diabetes.

In our present invention we explore new compounds having antidiabetic activity, we propose to synthesize new compounds containing substituted pyridine systems.

With an objective of providing compounds, which are effective for such treatments as well as for the treatment of, for example, insulin resistance, hyperlipidemia, obesity, we have continued our research to develop new thiazoldinediones along with other heterocyclic analogs.

Few prior art references, which disclose the closest compounds, are given here:

i) US 2004/0142991 discloses compounds of formula (I)

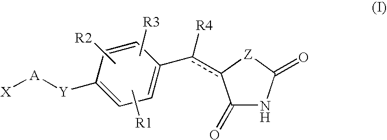

wherein ---- represents optional double bond; Y represents oxygen, sulfur or NR, wherein R represents hydrogen or alkyl; Z represents oxygen or sulfur; R₁, R₂, R₃ and R₄ may be same or different and independently represent hydrogen, halogen, hydroxy, nitro, cyano, formyl, amino, alkyl, alkoxy group; A represents a bond or substituted or unsubstituted aryl, heterocyclyl or heteroaryl ring; X represents amino acid or its derivatives

The compounds of this formula is shown in example (1)

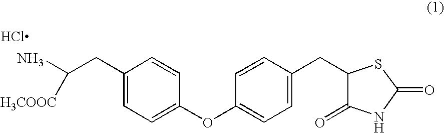

ii) U.S. Pat. No. 4,687,777 discloses compounds of formula (I) and thiazolidinedione derivatives of the formula I and pharmacologically acceptable salts thereof are novel compounds, which exhibit in mammals blood sugar- and lipid-lowering activity, and are of value as a therapeutic agent for treatment of diabetes and hyperlipemia.

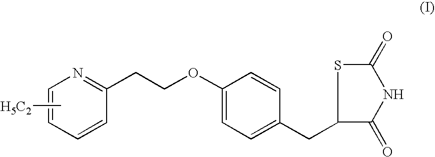

iii) Tetrahedron asymmetry 14, 2003, 2619-2623 discloses the four step synthesis of 4-(4-hydroxybenzyl)-oxazolidin-2-one (S-1) from N-Boc-L-tyrosine and discloses the intermediate of formula (S-1). The N-substituted derivatives of S-1 used were synthesized by conventional methods.

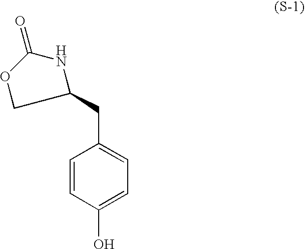

### OBJECTIVE OF THE INVENTION

With an objective to develop novel compounds for lowering blood glucose, free fatty acids, cholesterol and triglyceride levels in type II diabetes, we focused our research to develop new compounds effective in the treatment of the above-mentioned diseases. Efforts in this direction have led to compounds having general formula (I).

The main objective of the present invention is therefore, to provide novel pyridine derivatives and their pharmaceutically acceptable salts.

Another objective of the present invention is to provide novel pyridine derivatives and their pharmaceutically acceptable salts that are useful for treatment of disorders associated with insulin resistance, such as polycystic ovary syndrome, as well as hyperlipidemia, coronary artery disease and peripheral vascular disease, and for the treatment of inflammation and immunological diseases, particularly those mediated by cytokines such as TNF-α, IL-1, IL-6, IL-1β and cyclooxygenases such as COX-2.

Another objective of the present invention is to provide novel pyridine derivatives and their pharmaceutically acceptable salts having enhanced activities, without toxic effects or with reduced toxic effects.

Yet another objective of the present invention is to provide a process for the preparation of novel pyridine derivatives of formula (I), their stereoisomers, and their pharmaceutically acceptable salts, and compositions.

### SUMMARY OF THE INVENTION

The present invention relates to novel pyridine derivatives of the general formula (I),

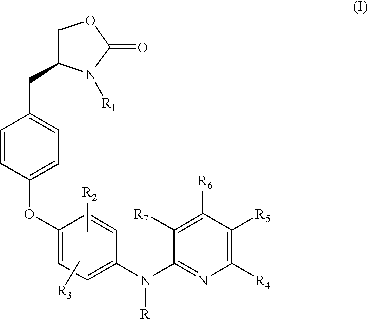

their stereoisomers, and their pharmaceutically acceptable salts, and compositions; wherein R and R₁ may be same or different and independently represent hydrogen, substituted or unsubstituted groups selected from alkyl, alkenyl, aryl, aryloxy, alkoxy, heteroaryl or heterocyclyl, COR₈; wherein R₈ represents substituted or unsubstituted groups selected from alkyl, alkenyl, aryl, aryloxy, alkoxy, heteroaryl or heterocyclyl; R₂ and R₃, may be same or different and independently represent hydrogen, halogen, hydroxy, nitro, cyano, formyl, amino, alkyl, haloalkyl, alkoxy group; R₄, R₅, R₆ and R₇ may be same or different and independently represents hydrogen, nitro, hydroxy, formyl, azido, halo, cyano or substituted or unsubstituted groups selected from alkyl, alkoxy, acyl, haloalkyl, amino, hydrazine, monoalkylamino, dialkylamino, acylamino, alkylsulfonyl, alkylsulfinyl, arylsulfonyl, arylsulfinyl, arylthio, carboxylic acid and its derivatives.

### DETAILED DESCRIPTION OF THE INVENTION

Suitable groups represented by R and R₁ may be same or different and independently represent hydrogen, alkyl, alkenyl, substituted or unsubstituted groups selected from (C₁-C₄) alkyl groups such as methyl, ethyl, propyl, isopropyl, n-butyl, isobutyl, t-butyl and the like; substituted or unsubstituted linear or branched (C₂-C₇) alkenyl groups such as ethenyl, propenyl, butenyl and the like; aryl groups such as phenyl, naphthyl and the like, the aryl group may be substituted; aryloxy, substituted or unsubstituted linear or branched (C₂-C₂₀) alkoxy groups such as methoxy, ethoxy, propoxy, n-butoxy, isobutoxy, t-butoxy and the like; heteroaryl groups such as pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl, benzothiazolyl, and the like, which may be substituted; heterocyclyl group such as pyrrolidinyl, thiazolidinyl, oxazolidinyl, morpholinyl, thiomorpholinyl, piperidinyl, piperazinyl, and the like, which may be substituted; COR₈, where R₈ represents substituted or unsubstituted groups selected from (C₁-C₄) alkyl group such as methyl, ethyl, propyl, isopropyl, n-butyl, isobutyl, t-butyl and the like; Substituted or unsubstituted linear or branched (C₂-C₇) alkenyl groups such as ethenyl, propenyl, butenyl and the like; aryl groups such as phenyl, naphthyl and the like, the aryl group may be substituted; aryloxy, substituted or unsubstituted linear or branched (C₂-C₂₀) alkoxy groups such as methoxy, ethoxy, propoxy, n-butoxy, isobutoxy, t-butoxy and the like;

Suitable groups represented by R₂ and R₃ are selected from hydrogen, halogens such as fluorine, chlorine, bromine or iodine; hydroxy, nitro, cyano, formyl, amino, unsubstituted linear or branched (C₁-C₄) alkyl groups such as methyl, ethyl, n-propyl, isopropyl, n-butyl, isobutyl, t-butyl and the like; haloalkyl groups such as chloromethyl, chloroethyl, trifluoromethyl, trifluoroethyl, dichloromethyl, dichloroethyl, trichloromethyl, difluoromethyl, and the like, which may be substituted; alkoxy groups such as methoxy, ethoxy, n-propoxy, isopropoxy and the like, which may be substituted; R₄, R₅, R₆ and R₇ may be same or different and independently represents hydrogen, halogens such as fluorine, chlorine, bromine or iodine; hydroxy, nitro, cyano, formyl, amino, azido, hydrazine; unsubstituted or unsubstituted groups selected from linear or branched (C₁-C₄) alkyl groups such as methyl, ethyl, n-propyl, isopropyl, n-butyl, isobutyl, t-butyl and the like; haloalkyl groups such as chloromethyl, chloroethyl, trifluoromethyl, trifluoroethyl, dichloromethyl, dichloroethyl, trichloromethyl, difluoromethyl, and the like, which may be substituted; alkoxy groups such as methoxy, ethoxy, n-propoxy, isopropoxy and the like, which may be substituted; monoalkylamino groups such as —NHCH₃, —NHC₂H₅, —NHC₃H₇—NHC₆H₁₃, and the like, which may be substituted; dialkylamino groups such as —N(CH₃)₂, —NCH₃(C₂H₅), —N(C₂H₅)₂ and the like, which may be substituted; carboxylic acids and its derivatives such as esters or amides; acylamino groups such as —NHC(═O)CH₃, —NHC(═O)C₂H₅, —NHC(═O)C₃H₇, —NHC(═O)C₆H₁₃, and the like, which may be substituted; alkylsulfonyl groups such as methylsulfonyl, ethylsulfonyl, n-propylsulfonyl, iso-propylsulfonyl and the like, the alkylsulfonyl group may be substituted; arylsulfonyl groups such as phenylsulfonyl or naphthylsulfonyl, the arylsulfonyl group may be substituted; alkylsulfinyl groups such as methylsulfinyl, ethylsulfinyl, n-propylsulfinyl, iso-propylsulfinyl and the like, the alkylsulfinyl group may be substituted; arylsulfinyl groups such as phenylsulfinyl or naphthylsulfinyl, the arylsulfinyl group may be substituted; alkylthio groups such as methylthio, ethylthio, n-propylthio, iso-propylthio and the like, the alkylthio group may be substituted; alkoxycarbonyl groups such as methoxycarbonyl, ethoxycarbonyl, n-propoxycarbonyl, isopropoxycarbonyl and the like.

Pharmaceutically acceptable salts of the present invention include alkali metal like Li, Na, and K, alkaline earth metals like Ca and Mg, salts of organic bases such as diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising of other solvents of crystallization such as alcohols.

The protecting groups P used in the invention are conventional protecting groups such as t-butoxy carbonyl (t-Boc), trityl, trifluoroacetyl, benzyloxy, benzyloxy carbonyl (Cbz) and the like.

### Particularly Useful Compounds According to the Invention Include:

1). (4S)-3-methyl-4-{4-[4-(pyridin-2-ylamino)phenoxy]benzyl}-1,3-oxazolidin-2-one.
2). (4S)4-(4-{4-[(5-nitropyridin-2-yl)amino]phenoxy}benzyl)-3-methyl-1,3-oxazolidin-2-one.
3). (4S)3-methyl-4-[4-(4-{[5-(trifluoromethyl)pyridin-2-yl]amino}phenoxy)benzyl]-1,3-oxazolidin-2-one.
4). (4S)-3-ethyl-4-{4-[4-(pyridin-2-ylamino)phenoxy]benzyl}-1,3-oxazolidin-2-one.
5). (4S)3-ethyl-4-[4-(4-{[5-(trifluoromethyl)pyridin-2-yl]amino}phenoxy)benzyl]-1,3-oxazolidin-2-one.
6). (4S)-4-[4-(2-fluoro-4-{[5-nitropyridin-2-yl]amino}phenoxy)benzyl]-3-ethyl-1,3-oxazolidin-2-one.
7). (4S)-4-[4-(2-fluoro-4-{[5-(trifluoromethyl)pyridin-2-yl]amino}phenoxy)benzyl]-3-ethyl-1,3-oxazolidin-2-one.
8). (4S)-4-[4-(2-fluoro-4-{[5-pyridin-2-yl]amino}phenoxy)benzyl]-3-ethyl-1,3-oxazolidin-2-one.
9). (4S)-4-[4-(2-fluoro-4-{[5-pyridin-2-yl]amino}phenoxy)benzyl]-3-methyl-1,3-oxazolidin-2-one.
10). (4S)-4-[4-(4-{[5-nitropyridin-2-yl]amino}phenoxy)benzyl]-3-ethyl-1,3-oxazolidin-2-one.
11). (4S)-4-[4-(2-fluoro-4-{[5-nitropyridin-2-yl]amino}phenoxy)benzyl]-3-methyl-1,3-oxazolidin-2-one.
12). (4S)-4-[4-(2-fluoro-4-{[3-nitropyridin-2-yl]amino}phenoxy)benzyl]-3-ethyl-1,3-oxazolidin-2-one.
13). 2-{[3-fluoro-4-(4-{[(4S)-3-ethyl-2-oxo-1,3-oxazolidin-4-yl]methyl}phenoxy)phenyl]amino}nicotinonitrile.
14). 2-[(4-{4-[(3-ethyl-2-oxo-1,3-oxazolidin-4-yl)methyl]phenoxy}phenyl)amino]nicotinonitrile.
15). 2-{[4-(4-{[(4S)-3-methyl-2-oxo-1,3-oxazolidin-4-yl]methyl}phenoxy)phenyl]amino}nicotinonitrile.
16). (4S)-3-methyl-4-{4-[4-(3-nitropyridin-2-ylamino)phenoxy]benzyl}-1,3-oxazolidin-2-one.
17). (4S)-4-[4-(2-fluoro-4-{[3-nitropyridin-2-yl]amino}phenoxy)benzyl]-3-methyl-1,3-oxazolidin-2-one.
18). (4S)-4-[4-(2-fluoro-4-{[5-(trifluoromethyl)pyridin-2-yl]amino}phenoxy)benzyl]-3-methyl-1,3-oxazolidin-2-one.
19). (4S)-3-ethyl-4-{4-[4-(3-nitropyridin-2-ylamino)phenoxy]benzyl}-1,3-oxazolidin-2-one.
20). (4S)-4-(4-{2-fluoro-4-[methyl(5-nitropyridin-2-yl)amino]phenoxy}benzyl)-3-methyl-1,3-oxazolidin-2-one.
21). (4S)-2-[methyl(4-{4-[(3-ethyl-2-oxo-1,3-oxazolidin-4-yl)methyl]phenoxy}phenyl)amino]nicotinonitrile.
22). 2-{[3-fluoro-4-(4-{[(4S)-3-methyl-2-oxo-1,3-oxazolidin-4-yl]methyl}phenoxy)phenyl]amino}nicotinonitrile.
23). (4S)-4-(4-{2-fluoro-4-[methyl(3-nitropyridin-2-yl)amino]phenoxy}benzyl)-3-methyl-1,3-oxazolidin-2-one.
24). (4S)-2-[ethyl(4-{4-[(3-ethyl-2-oxo-1,3-oxazolidin-4-yl)methyl]phenoxy}phenyl)amino]nicotinonitrile.
25). (4S)-4-(4-{4-[methyl(3-nitropyridin-2-yl)amino]phenoxy}benzyl)-3-ethyl-1,3-oxazolidin-2-one.
26). (4S)-4-(4-{4-[ethyl(3-nitropyridin-2-yl)amino]phenoxy}benzyl)-3-ethyl-1,3-oxazolidin-2-one.
27). (4S)-4-(4-{2-fluoro-4-[ethyl(5-nitropyridin-2-yl)amino]phenoxy}benzyl)-3-methyl-1,3-oxazolidin-2-one.
28). (4S)-4-(4-{4-[methyl(5-nitropyridin-2-yl)amino]phenoxy}benzyl)-3-ethyl-1,3-oxazolidin-2-one.
29). (4S)-4-(4-{4-[ethyl(5-nitropyridin-2-yl)amino]phenoxy}benzyl)-3-ethyl-1,3-oxazolidin-2-one.
30). 2-[(3-fluoro-4-{4-[(4S)-3-methyl-2-oxo-1,3-oxazolidin-4-yl)methyl]phenoxy}phenyl)(methyl)amino]nicotinonitrile.
31). (4S)-4-(4-{4-[methyl(3-nitropyridin-2-yl)amino]phenoxy}benzyl)-3-methyl-1,3-oxazolidin-2-one.
32). (4S)-4-(4-{4-[ethyl(3-nitropyridin-2-yl)amino]phenoxy}benzyl)-3-methyl-1,3-oxazolidin-2-one.
33). (4S)-4-(4-{4-[methyl(5-nitropyridin-2-yl)amino]phenoxy}benzyl)-3-methyl-1,3-oxazolidin-2-one.
34). (4S)-4-(4-{2-fluoro-4-[methyl(5-(trifluoromethyl)pyridin-2-yl)amino]phenoxy}benzyl)-3-methyl-1,3-oxazolidin-2-one.
35). (4S)-4-(4-{4-[ethyl(5-nitropyridin-2-yl)amino]phenoxy}benzyl)-3-methyl-1,3-oxazolidin-2-one.
36). (4S)-4-(4-{2-fluoro-4-[ethyl(5-nitropyridin-2-yl)amino]phenoxy}benzyl)-3-ethyl-1,3-oxazolidin-2-one.
37). (4S)-4-(4-{2-fluoro-4-[ethyl(pyridin-2-yl)amino]phenoxy}benzyl)-3-ethyl-1,3-oxazolidin-2-one.
38). (4S)-4-(4-{2-fluoro-4-[methyl(5-(trifluoromethyl)pyridin-2-yl)amino]phenoxy}benzyl)-3-ethyl-1,3-oxazolidin-2-one.
39). 2-[(3-fluoro-4-{4-[(4S)-3-ethyl-2-oxo-1,3-oxazolidin-4-yl)methyl]phenoxy}phenyl)(methyl)amino]nicotinonitrile.
40). 2-[(3-fluoro-4-{4-[(4S)-3-ethyl-2-oxo-1,3-oxazolidin-4-yl)methyl]phenoxy}phenyl)(ethyl)amino]nicotinonitrile.
41). (4S)-4-(4-{2-fluoro-4-[ethyl(3-nitropyridin-2-yl)amino]phenoxy}benzyl)-3-ethyl-1,3-oxazolidin-2-one.
42). (4S)-4-(4-{4-[methyl(pyridin-2-yl)amino]phenoxy}benzyl)-3-ethyl-1,3-oxazolidin-2-one.
43). (4S)-4-(4-{2-fluoro-4-[methyl(5-nitropyridin-2-yl)amino]phenoxy}benzyl)-3-ethyl-1,3-oxazolidin-2-one.
44). (4S)-4-(4-{2-fluoro-4-[ethyl(5-(trifluoromethyl)pyridin-2-yl)amino]phenoxy}benzyl)-3-ethyl-1,3-oxazolidin-2-one.
45). (4S)-2-[methyl(4-{4-[(3-methyl-2-oxo-1,3-oxazolidin-4-yl)methyl]phenoxy}phenyl)amino]nicotinonitrile.
46). (4S)-2-[ethyl(4-{4-[(3-methyl-2-oxo-1,3-oxazolidin-4-yl)methyl]phenoxy}phenyl)amino]nicotinonitrile.
47). 2-[(3-fluoro-4-{4-[(4S)-3-methyl-2-oxo-1,3-oxazolidin-4-yl)methyl]phenoxy}phenyl)(ethyl)amino]nicotinonitrile.
48). (4S)-4-(4-{2-fluoro-4-[ethyl(3-nitropyridin-2-yl)amino]phenoxy}benzyl)-3-methyl-1,3-oxazolidin-2-one.
49). (4S)-4-(4-{2-fluoro-4-[methyl(3-nitropyridin-2-yl)amino]phenoxy}benzyl)-3-ethyl-1,3-oxazolidin-2-one.
50). (4S)-4-(4-{4-[methyl(pyridin-2-yl)amino]phenoxy}benzyl)-3-methyl-1,3-oxazolidin-2-one.
51). (4S)-4-(4-{2-fluoro-4-[ethyl(pyridin-2-yl)amino]phenoxy}benzyl)-3-methyl-1,3-oxazolidin-2-one.

Preferred salts for the list of compounds above are hydrochloride, hydrobromide, sodium, potassium or magnesium.

According to another feature of the present invention, there is provided a process for the preparation of compounds of formula (I), wherein all other symbols are as defined earlier, as shown below in the scheme-1, wherein X represents halogen and all other groups are as defined earlier.

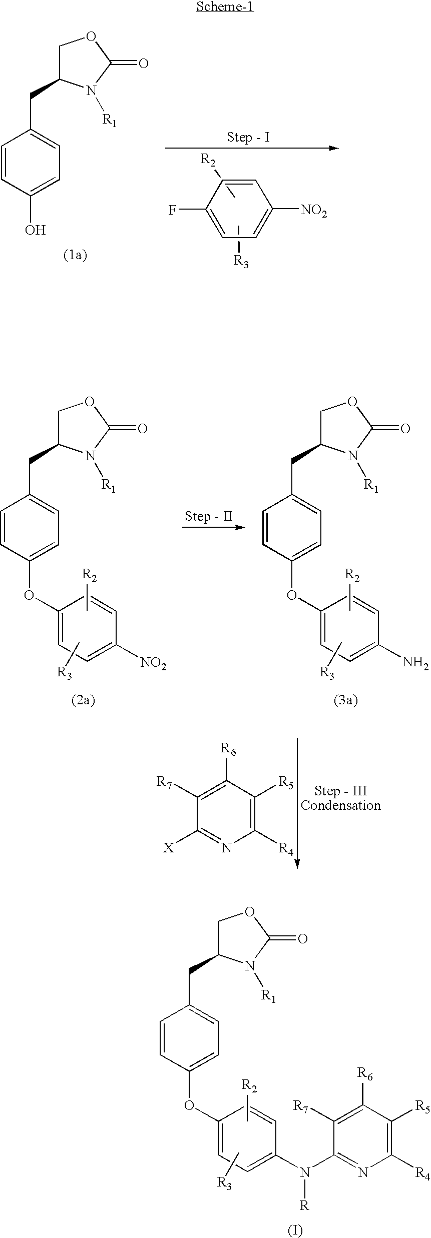

### The Compound of the General Formula (I), are Prepared by the Following Procedure:

Step-(I): Condensation of the compound of formula (1a), with halo nitro benzene was carried out in the presence of solvents selected from toluene, DMF, tetrahydrofuran, chloroform, dichloromethane, dichloroethane, ethyl acetate, o-dichlorobenzene or a mixture thereof, in the presence of a base such as triethyl amine, diethylamine, pyridine, alkali hydroxides, alkaline earth metal hydroxide, alkali carbonates such as sodium hydroxide, potassium hydroxide, potassium carbonate and the like to get the compound of formula (2a). The reaction is carried out at a temperature in the range of room temperature to reflux temperature preferably 60° C. to 100° C. The compound of the formula (Ia) is prepared according to the procedure described in Tetrahedron asymmetry 14, 2003, 2619-2623.

Step-(II): Hydrogenation of the compound of the formula (2a) by using catalysts such as Raney nickel or Pd/C and the like is carried out in the presence of solvents such as methanol, ethanol, ethylacetate, n-butylacetate or a mixture thereof. The reaction may be carried out at 30° C. to 50° C. The duration of the reaction may range from 2 to 6 hours, to produce a compound of the formula (3a).

Step-(III): The compound of formula (3a) is reacted with halo pyridine in the presence of solvents such as toluene, methanol, ethanol, tetrahydrofuran, chloroform, dichloromethane, dichloroethane, ethylacetate, o-dichlorobenzene or a mixture thereof. The reaction may be carried out at 50° C. to 150° C. The duration of the reaction may range from 2 to 24 hours, to produce a compound of formula (I).

Alternatively whenever R₁ is in the protected form (the protecting groups are as defined earlier) then the final step involves an additional deprotection step, wherein the protecting group is removed by using acids such as HCl, sulfuric acid, acetic acid, trifluoroacetic acid and the like, in the presence of solvents such as dichloromethane, ethyl acetate, water and the like or mixture thereof at a temperature in the range of −10° C. to 50° C. to furnish the compound of the general formula (I).

It is appreciated that in any of the above-mentioned reactions, any reactive group in the substrate molecule may be protected according to the conventional chemical practice. Suitable protecting groups in any of the above-mentioned reactions are those used conventionally in the art. The methods of formation and removal of such protecting groups are those conventional methods appropriate to the molecule being protected.

The pharmaceutically acceptable salts are prepared by reacting the compound of formula (I) with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixtures of solvents may be used. Organic bases like lysine, arginine, diethanolamine, choline, guanidine and their derivatives etc. may also be used. Alternatively, acid addition salts are prepared by treatment with acids such as hydrochloric acid, hydrobromic acid, nitric acid, sulfuric acid, phosphoric acid, p-toluenesulfonic acid, methanesulfonic acid, acetic acid, citric acid, maleic acid, salicylic acid, hydroxynaphthoic acid, ascorbic acid, palmitic acid, succinic acid, benzoic acid, benzene sulfonic acid, tartaric acid and the like in solvents like ethyl acetate, ether, alcohols, acetone, THF, dioxane etc. Mixture of solvents may also be used.

The pharmaceutical composition may be in the forms normally employed, such as tablets, capsules, powders, syrups, solutions, aerosols, suspensions and the like, may contain flavoring agents, sweeteners etc. in suitable solid or liquid carriers or diluents, or in suitable sterile media to form injectable solutions or suspensions. Such compositions typically contain from 1 to 20%, preferably 1 to 10% by weight of the active compound, the remainder of the composition being the pharmaceutically acceptable carriers, diluents or solvents.

The invention is explained in detail in the examples given below which are provided by way of illustration only and therefore should not be construed to limit the scope of the invention.

### Example 1

### Preparation of 3-methyl-4-{4-[4-(pyridin-2-ylamino)phenoxy]benzyl}-1,3-oxazolidin-2-one

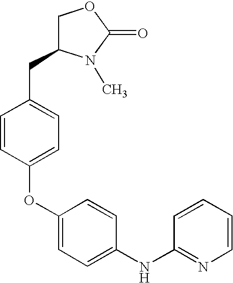

### Stage-I

### Synthesis of 3-methyl-4-[4-(4-nitrophenoxy)benzyl]-1,3-oxazolidin-2-one

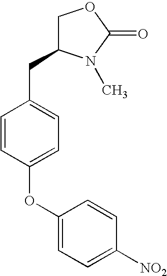

To a solution of 4-(4-hydroxybenzyl)-3-methyl-1,3-oxazolidin-2-one (4.0 g, 19.32 mmol) in dimethylformamide (40 ml), and potassium carbonate (26.6 g, 193.2 mmol) was charged 4-fluoro nitrobenzene (3.27 g, 23.18 mmol). The reaction mixture was heated to 80° C. for 5 hours and was subsequently quenched with cold water (150 ml) and extracted with ethyl acetate. The solvent was evaporated to give the desired product 6.3 g (99.5%), ¹HNMR [CDCl₃, 400 MHz] δ ppm 2.77 (q, 1H), 2.93 (s, 3H)), 3.15 (dd, 1H), 3.95 (m, 1H), 4.02 (m, 1H), 4.25 (t, 1H), 7.00 (dd, 2H), 7.07 (dd, 2H), 7.24 (m, 2H), and 8.21 (d, 2H); m/z^{M+1}: 329.

### Stage-II

### Synthesis of 4-[4-(4-aminophenoxy)benzyl]-3-methyl-1,3-oxazolidin-2-one

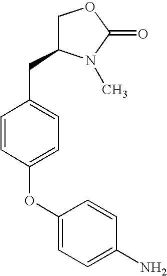

10% Pd/C (0.22 g) was added to the solution of 3-methyl-4-[4-(4-nitrophenoxy)benzyl]-1,3-oxazolidin-2-one (4.0 g, 12.19 mmol) in methanol (200 ml) and the reaction mixture was hydrogenated at 40 psi for 4 hours. After completion of reaction the catalyst was filtered and the reaction mixture was concentrated to gave 4-[4-(4-aminophenoxy)benzyl]-3-methyl-1,3-oxazolidin-2-one. 3.3 g (90.0%); ¹HNMR [CDCl₃, 400 MHz] δ ppm 2.66 (q, 1H), 2.89 (s, 3H), 3.06 (dd, 1H), 3.7 (bs, 2H), 3.87 (m, 1H), 3.98 (q, 1H), 4.20 (t, 1H), 6.68 (dd, 2H), 6.87 (m, 4H), and 7.16 (d, 2H); m/z^{M+1}: 299.

### Stage-III

### Synthesis of 3-methyl-4-{4-[4-(pyridin-2-ylamino)phenoxy]benzyl}-1,3-oxazolidin-2-one

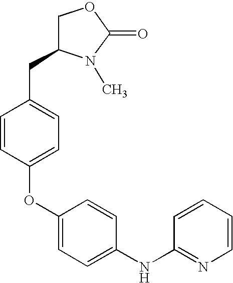

4-[4-(4-aminophenoxy)benzyl]-3-methyl-1,3-oxazolidin-2-one (1.0 g, 3.35 mmol) and 2-chloro pyridine (1.9 ml, 20.17 mmol) were stirred under a nitrogen atmosphere at 130° C. for 20 hours. After completion of reaction the reaction mixture was quenched with cold water and was extracted with ethylacetate. The solvent was evaporated to give the crude product, which was purified by column chromatography to give the desired product (0.5 g, 40%); ¹HNMR. [CDCl₃, 400 MHz] δ 2.70 (q, 1H), 2.90 (s, 3H), 3.10 (dd, 1H), 3.90 (m, 1H), 4.00 (q, 1H), 4.22 (t, 1H), 6.78 (m, 2H), 7.00 (m, 4H), 7.09 (d, 2H), 7.35 (dd, 2H), 7.49 (dd, 1H), and 8.19 (d, 1H); m/z^{M+1} 376.1.

The Following Compounds were Prepared According to the Procedure Given in Example 1.

| Exp. | | |
|---|---|---|
| No. | Structure | Analytical data |
| 2. | 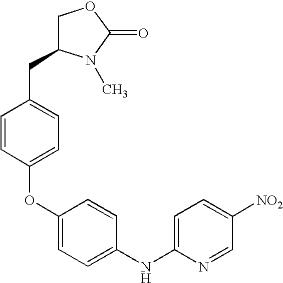 | Yield (0.49 g); ¹HNMR. [CDCl₃, 400 MHz] δ 2.73 (m, 1 H), 2.91 (s, 3 H), 3.09 (dd, 1 H), 3.92 (m, 1 H), 4.01 (q, 1 H), 4.23 (t, 1 H), 6.70 (d, 1 H), 7.02 (m, 4 H), 7.14 (d, 2 H), 7.37 (d, 2 H), 8.25 (d, 1 H), 9.08 (s, 1 H); m/z^{M+1}: 421.1 |
| 3. | 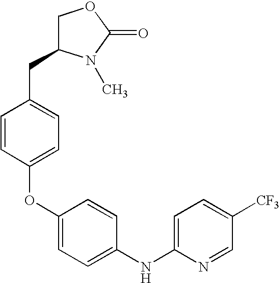 | Yield (0.8 g); ¹HNMR. [CDCl₃, 400 MHz] δ 2.71 (m, 1 H), 2.91 (s, 3 H), 3.10 (dd, 1 H), 3.91 (m, 1 H), 4.00 (dd, 1 H), 4.22 (dd, 1 H), 6.75 (d, 1 H), 6.89 (s, 1 H), 7.02 (m, 4 H), 7.12 (d, 2 H), 7.35 (d, 2 H), 7.64 (s, 1 H), 8.42 (s, 1 H); m/z^{M+1}: 444.1 |
| 4. | 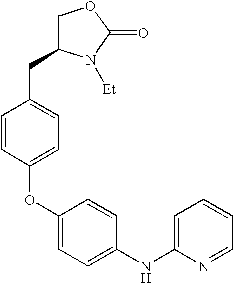 | Yield (0.5 g); ¹HNMR. [CDCl₃, 400 MHz] δ 1.20 (t, 3 H), 2.67 (q, 1 H), 3.15 (m, 2 H), 3.60 (q, 1 H), 4.01 (q, 2 H), 4.18 (t, 1 H), 6.54 (s, 1 H), 6.76 (m, 2 H), 6.97 (m, 4 H), 7.10 (d, 2 H), 7.34 (d, 2 H), 7.49 (t, 1 H), 8.20 (s, 1 H); m/z^{M+1}: 390.1 |
| 5. | 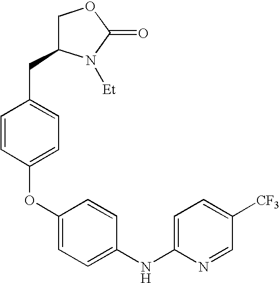 | Yield (0.6 g); ¹HNMR. [DMSO-d₆, 400 MHz] δ 1.08 (t, 3 H), 2.67 (m, 1 H), 3.00 (dd, 1 H), 3.13 (m, 1 H), 3.37 (m, 1 H), 3.95 (m, 1 H), 4.00 (m, 1 H), 4.15 (t, 1 H), 6.90 (t, 3 H), 7.01 (d, 2 H), 7.26 (d, 2 H), 7.68 (d, 2 H), 7.82 (dd, 1 H). 8.45 (s, 1 H), 9.60 (s, 1H); m/z^{M+1} 458.1 |
| 6. | 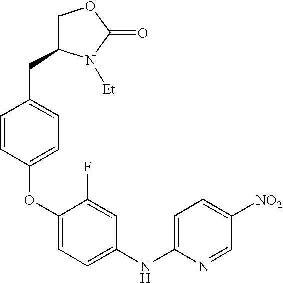 | Yield (0.18 g); ¹HNMR. [CDCl₃, 400 MHz] δ 1.20 (t, 3 H), 2.67 (m, 1 H), 3.09 (dd, 1 H), 3.17 (m, 1 H), 3.60 (m, 1 H), 3.99 (t, 2 H), 4.19 (t, 1 H), 6.76 (d, 2 H), 6.95 (d, 2 H), 7.13 (m. 4 H), 7.54 (dd, 1 H), 8.30 (d, 1 H), 9.12 (s. 1 H); m/z^{M+1} 453.1 |
| 7. | 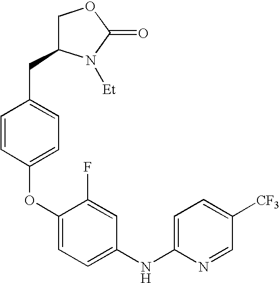 | Yield (0.60 g); ¹HNMR. [CDCl₃, 400 MHz] δ 1.19 (t, 3 H), 2.66 (q, 1 H), 3.07 (m, 1 H), 3.16 (m, 1 H), 3.60 (m, 1 H), 4.00 (q, 2 H), 4.18 (t, 1 H), 6.81 (d, 1 H), 6.93 (d, 2 H), 7.09 (m. 4 H), 7.51 (dd, 1 H), 7.70 (d, 1 H), 8.47 (s, 1 H); m/z^{M+1} 476.1 |
| 8. | 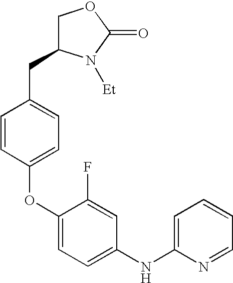 | Yield (0.50 g); ¹HNMR. [CDCl₃, 400 MHz] δ 1.19 (t, 3 H), 2.64 (q, 1 H), 3.12 (m, 2 H), 3.59 (m, 1 H), 3.98 (q, 2 H), 4.17 (t, 1 H), 6.53 (s, 1 H), 6.79 (d, 2 H), 6.92 (d, 2 H) 7.05 (m, 4 H), 7.50 (m, 2 H), 8.23 (d, 1 H); m/z^{M+1} 408.1 |
| 9. | 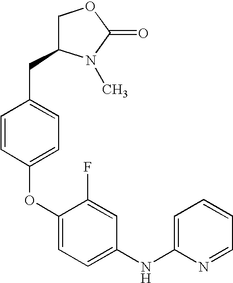 | Yield (0.40 g); ¹HNMR. [CDCl₃, 400 MHz] δ 2.67 (q, 1 H), 2.89 (s, 3 H), 3.10 (dd, 1 H), 3.89 (m, 1 H), 3.98 (t, 1 H), 4.20 (t, 1 H), 6.56 (s, 1 H), 6.79 (t, 2 H), 6.92 (d, 2 H), 7.08 (m, 4 H), 7.53 (m, 2 H), 8.23 (d, 1 H) m/z^{M+1} 394.1 |
| 10. | 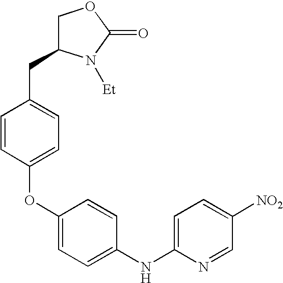 | Yield (0.180 g); ¹HNMR. [CDCl₃, 400 MHz] δ 1.23 (t, 3 H), 2.70 (q, 1 H), 3.15 (m, 2 H), 3.61 (m, 1 H), 4.03 (q, 2 H), 4.20 (t, 1 H) 6.70 (d, 1 H), 7.02 (m, 4 H), 7.14 (d, 2 H), 7.37 (d, 2 H), 8.24 (dd, 1 H), 9.08 (s, 1 H); m/z^{M+1} 435.1 |
| 11. | 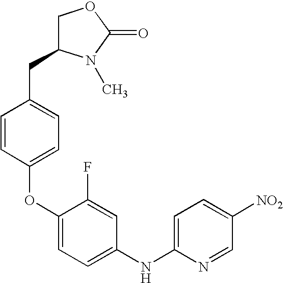 | Yield (0.13 g); ¹HNMR. [CDCl₃, 400 MHz] δ 2.74 (q, 1 H), 2.91 (s, 3 H), 3.07 (dd, 1 H), 3.98 (m, 1 H), 4.0 (m, 1 H), 4.21 (t, 1 H), 6.76 (d, 1 H), 6.95 (d, 2 H), 7.14 (m, 4 H), 7.53 (d, 1 H), 8.30 (d, 1 H), 9.12 (s, 1 H); m/z^{M+1} 439.1 |
| 12. | 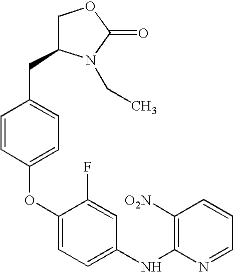 | Yield (0.40 g); ¹HNMR. [CDCl₃, 400 MHz] δ 1.19 (t, 3 H), 2.66 (q, 1 H), 3.14 (m, 2 H), 3.60 (m, 1 H), 4.00 (q, 2 H), 4.18 (t, 1 H), 6.90 (m, 1 H), 6.94 (d, 2 H), 7.11 (m, 3 H), 7.25 (m, 1 H) 7.84 (d, 1 H), 8.55 (t, 2 H), 10.17 (s, 1 H); m/z^{M+1} 453.1 |
| 13. | 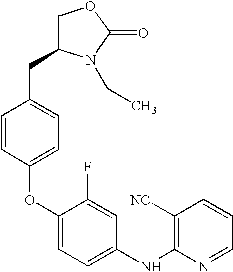 | Yield (0.56 g); ¹HNMR. [CDCl₃, 400 MHz] δ 1.19 (t, 3 H), 2.65 (q, 1 H), 3.14 (m, 2 H), 3.59 (m, 1 H), 4.00 (q, 2 H), 4.18 (t, 1 H), 6.86 (dd, 1 H), 6.94 (d, 2 H), 7.03 (d, 1 H), 7.09 (t, 2 H), 7.10 (d, 1 H), 7.82 (t, 2 H), 8.41 (d, 1 H); m/z^{M+1} 433.1 |
| 14. | 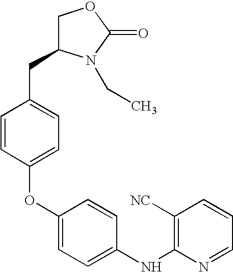 | Yield (0.15 g); ¹HNMR. [CDCl₃, 400 MHz] δ 1.20 (t, 3 H), 2.66 (m, 1 H), 3.10 (dd, 1 H), 3.17 (m, 1 H), 3.59 (m, 1 H), 4.02 (q, 2 H), 419 (t, 1 H), 6.78 (m, 1 H), 7.0 (m, 5 H), 7.12 (m, 2 H), 7.54 (d, 2 H), 7.78 (dd, 1 H), 8.36 (d, 1 H); m/z^{M+1} 415.1 |
| 15. | 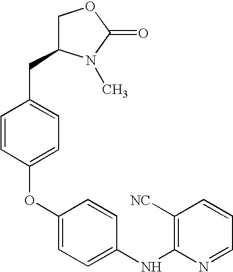 | Yield (0.21 g); ¹HNMR. [CDCl₃, 400 MHz] δ 2.70 (q, 1 H), 2.91 (s, 3 H), 3.10 (dd, 1 H), 3.91 (m, 1 H), 4.00 (t, 1 H), 4.22 (t, 1 H), 6.79 (m. 1 H), 6.99 (m, 4 H), 7.12 (d, 2 H), 7.54 (d, 2 H), 7.78 (d, 1 H), 8.36 (d, 1 H); m/z^{M+1} 401.1 |
| 16. | 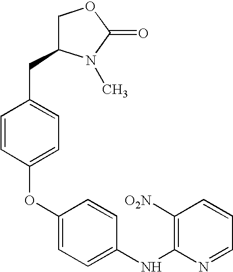 | Yield (0.25 g); ¹HNMR. [CDCl₃, 400 MHz] δ 2.69 (q, 1 H), 2.90 (s, 3 H), 3.10 (dd, 1 H), 3.90 (m, 1 H), 4.00 (q, 1 H), 4.22 (t, 1 H), 6.82 (d. 1 H), 7.02 (m, 4 H), 7.12 (d, 2 H), 7.59 (d, 2 H), 8.46 (d, 1 H), 8.52 (d, 1 H), 10.07 (s, 1 H); m/z^{M+1} 421.1 |
| 17. | 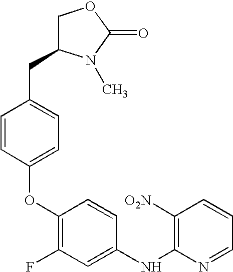 | Yield (1.15 g); ¹HNMR. [CDCl₃, 400 MHz] δ 2.69 (q, 1 H), 2.90 (s, 3 H), 3.11 (dd, 1 H), 3.90 (m, 1 H), 3.98 (t, 1 H), 4.21 (t, 1 H), 6.90 (m. 1 H), 6.94 (m, 2 H), 7.10 (m, 3 H), 7.26 (d, 1 H), 7.85 (dd, 1 H), 8.53 (m, 2 H), 10.17 (s, 1 H); m/z^{M+1} 439.1 |
| 18. | 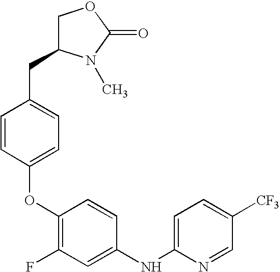 | Yield (1.70 g); ¹HNMR. [CDCl₃, 400 MHz] δ 2.69 (q, 1 H), 2.90 (s, 3 H), 3.09 (dd, 1 H), 3.89 (m, 1 H), 3.98 (t, 1 H), 4.21 (dd, 1 H), 6.81 (d. 1 H), 6.94 (d, 3 H), 7.10 (m, 4 H), 7.51 (dd, 1 H), 7.70 (dd, 1 H), 8.47 (s, 1 H); m/z^{M+1} 462.1 |
| 19. | 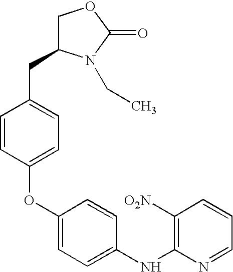 | Yield (1.39 g); ¹HNMR. [CDCl₃, 400 MHz] δ 1.20 (t, 3 H), 2.67 (t, 1 H), 3.16 (m, 2 H), 3.61 (m, 1 H), 4.02 (q, 2 H), 4.19 (t, 1 H), 6.83 (m, 1 H), 7.02 (m, 4 H), 7.13 (d, 2 H), 7.59 (d, 2 H), 8.50 (dd, 2 H), 10.07 (s, 1 H); m/z^{M+1} 435.1 |
| 20. | 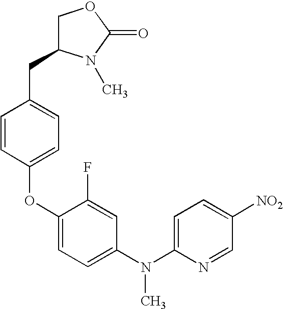 | Yield (0.34); ¹HNMR. [CDCl₃, 400 MHz] δ 2.75 (q, 1 H), 2.90 (s, 3 H), 3.08 (dd, 1 H), 3.57 (s, 3 H), 3.98 (m, 1 H), 4.00 (m, 1 H), 4.23 (t, 1 H), 6.43 (d, 1 H), 7.05 (m, 3 H), 7.15 (m, 4 H), 8.11 (dd, 1 H), 9.12 (d, 1 H); m/z^{M+1} 453.1 |
| 21. | 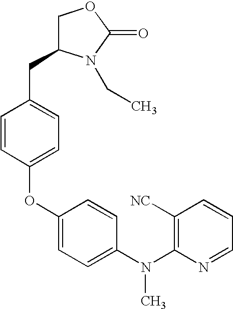 | Yield (0.35); ¹HNMR. [CDCl₃, 400 MHz] δ 1.19 (t, 3 H), 2.66 (q, 1 H), 3.10 (m, 2 H), 3.50 (s, 3 H), 3.60 (m, 1 H), 4.00 (q, 2 H), 4.18 (t, 1 H), 6.72 (m, 1 H), 7.07 (m, 4 H), 7.12 (d, 2 H), 7.24 (d, 2 H), 7.66 (dd, 1 H), 8.40 (d, 1 H); m/z^{M+1} 429.1 |
| 22. | 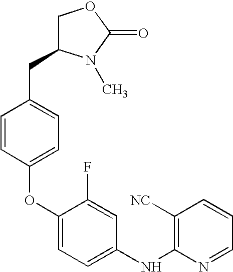 | Yield (0.81); ¹HNMR. [CDCl₃, 400 MHz] δ 2.69 (q, 1 H), 2.90 (s, 3 H), 3.07 (dd, 1 H), 3.89 (m, 1 H), 3.98 (m, 1 H), 4.21 (t, 1 H), 6.86 (t, 1 H), 6.94 (d, 2 H), 7.04 (s,1 H), 7.09 (m, 3 H), 7.10 (dd, 1 H), 7.83 (m, 2 H), 8.41 (d, 1 H); m/z^{M+1} 419.1 |
| 23. | 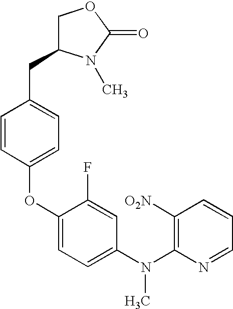 | Yield (1.15); ¹HNMR. [CDCl₃, 400 MHz] δ 2.68 (q, 1 H), 2.89 (s, 3 H), 3.09 (dd, 1 H), 3.59 (s, 3 H), 3.89 (m, 1 H), 3.98 (q, 1 H), 4.21 (t, 1 H), 6.79 (dd, 1 H), 6.94 (m, 5 H), 7.11 (d, 2 H), 8.01 (dd, 1 H), 8.48 (s, 1 H); m/z^{M+1} 453.1 |
| 24. | 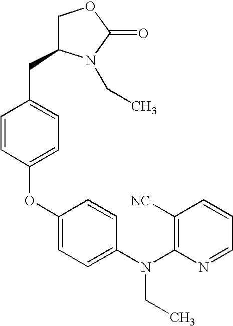 | Yield (0.25 g); ¹HNMR. [CDCl₃, 400 MHz] δ 1.20 (t, 3 H), 1.24 (t, 3 H), 267 (q, 1 H), 3.16 (m, 2 H), 3.59 (m, 1 H), 4.01 (m, 4 H), 4.18 (t, 1 H), 6.67 (d, 1 H), 7.07 (m, 6 H), 7.23 (m, 2 H), 7.62 (dd, 1 H), 8.36 (s, 1 H); m/z^{M+1} 443.1 |
| 25. | 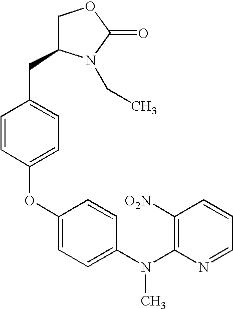 | Yield (0.40 g); ¹HNMR. [CDCl₃, 400 MHz] δ 1.20 (t, 3 H), 2.65 (q, 1 H), 3.08 (m, 1 H), 3.15 (m, 1 H), 3.57 (s, 3 H), 3.61 (m, 1 H) 4.01 (q, 2 H), 4.18 (t, 1 H), 6.85 (d, 1 H), 6.94 (t, 4 H), 7.09 (d, 2 H), 7.12 (d, 2 H), 7.94 (dd, 1 H), 8.44 (d, 1 H); m/z^{M+1} 449.1 |
| 26. | 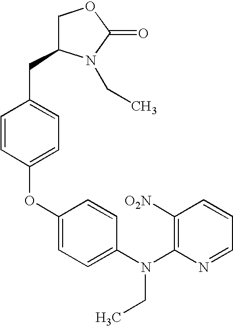 | Yield (0.40 g); ¹HNMR. [CDCl₃, 400 MHz] δ 1.20 (t, 3 H), 1.26 (t, 3 H), 2.72 (m, 1 H), 3.15 (m, 2 H), 3.59 (m, 1 H), 4.00 (q, 2 H), 4.15 (m, 3 H), 6.80 (dd, 1 H), 6.94 (m, 4 H), 7.00 (d, 2 H), 7.13 (d, 2 H), 7.90 (dd, 1 H), 8.41 (d, 1 H); m/z^{M+1} 463.1 |
| 27. | 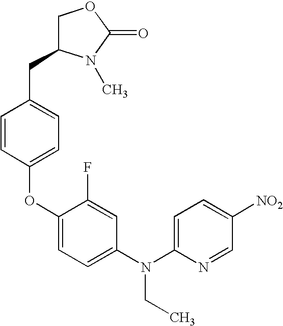 | Yield (0.35 g); ¹HNMR. [CDCl₃, 400 MHz] δ 1.27 (t, 3 H), 2.76 (q, 1 H), 2.91 (s, 3 H), 3.08 (dd, 1 H), 3.93 (m, 1 H), 4.00 (m, 1 H), 4.08 (q, 2 H), 4.23 (t, 1 H), 6.28 (d, 1 H), 7.03 (m, 3 H), 7.11 (m, 2 H), 7.17 (d, 2 H), 8.08 (dd, 1 H), 9.10 (s, 1 H); m/z^{M+1} 467.1 |
| 28. | 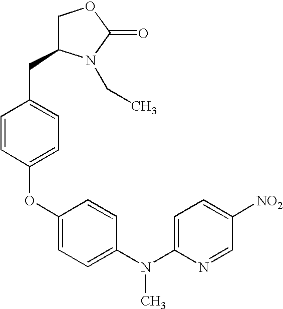 | Yield (0.25 g); ¹HNMR. [CDCl₃, 400 MHz] δ 1.21 (t, 3 H), 2.72 (q, 1 H), 3.17 (m, 2 H), 3.56 (s, 3 H), 3.62 (m, 1 H), 4.02 (q, 2 H), 4.21 (t, 1 H), 6.34 (d, 1 H), 7.07 (m, 4 H), 7.20 (m, 4 H), 8.04 (dd, 1 H), 9.12 (s, 1 H) m/z^{M+1} 449.1 |
| 29. | 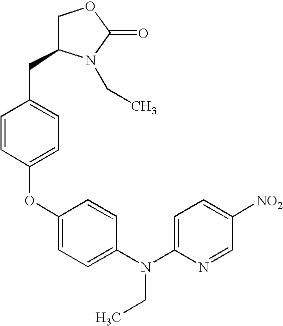 | Yield (0.15 g); ¹HNMR. [CDCl₃, 400 MHz] δ 1.22 (t, 3 H), 1.26 (t, 3 H), 2.72 (m, 1 H), 3.07 (dd, 1 H), 3.18 (m, 1 H), 3.62 (m, 1 H), 4.04 (m, 4 H), 4.20 (t, 1 H), 6.19 (d, 1 H), 7.07 (m, 4 H), 7.18 (m, 4 H), 8.02 (dd, 1 H), 9.10 (s, 1 H); m/z^{M+1} 463.1 |
| 30. | 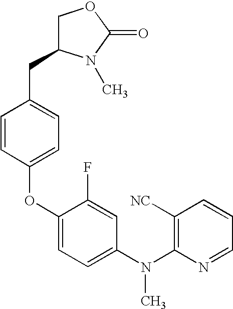 | Yield (0.30 g); ¹HNMR. [CDCl₃, 400 MHz] δ 2.67 (m, 1 H), 2.89 (s, 3 H), 3.09 (dd, 1 H), 3.52 (s, 3 H), 3.94 (m, 1 H), 3.98 (m, 1 H), 4.20 (t, 1 H), 6.80 (m, 1 H), 7.04 (m, 3 H), 7.10 (m, 4 H), 7.70 (dd, 1 H), 8.42 (d, 1 H), m/z^{M+1} 433.1 |
| 31. | 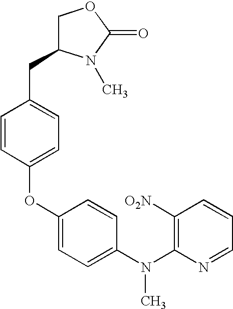 | Yield (0.50 g); ¹HNMR. [CDCl₃, 400 MHz] δ 2.68 (m, 1 H), 2.95 (s, 3 H), 3.09 (dd, 1 H), 3.57 (s, 3 H), 3.99 (m, 1 H), 4.0 (m, 1 H) 4.22 (t, 1 H), 6.85 (m, 1 H), 6.94 (m, 4 H), 7.03 (d, 2 H), 7.12 (d, 2 H), 7.94 (dd, 1 H), 8.45 (d, 1 H), m/z^{M+1} 435.1 |
| 32. | 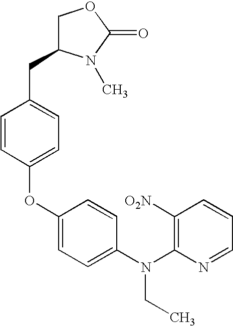 | Yield (0.40 g); ¹HNMR. [CDCl₃, 400 MHz] δ 1.26 (t, 3 H), 2.70 (m, 1 H), 2.91 (s, 3 H), 3.08 (dd, 1 H), 3.90 (m, 1 H), 4.00 (m, 1 H), 4.13 (q, 2 H), 4.21 (t, 1 H), 6.79 (m, 1 H), 6.94 (m, 4 H), 7.01 (d, 2 H), 7.12 (d, 2 H), 7.90 (dd, 1 H), 8.40 (d, 1 H); m/z^{M+1} 449.1 |
| 33. | 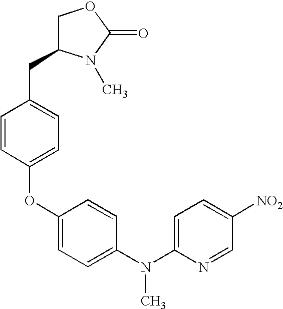 | Yield (0.40 g); ¹HNMR. [CDCl₃, 400 MHz] δ 2.76 (q, 1 H), 2.92 (s, 3 H), 3.11 (dd, 1 H), 3.56 (s, 3 H), 3.93 (m, 1 H), 4.01 (m, 1 H), 4.24 (t, 1 H), 6.34 (d, 1 H), 7.06 (m, 4 H), 7.21 (m, 4 H), 8.04 (dd, 1 H), 9.12 (s, 1 H), m/z^{M+1} 435.46 |
| 33. | 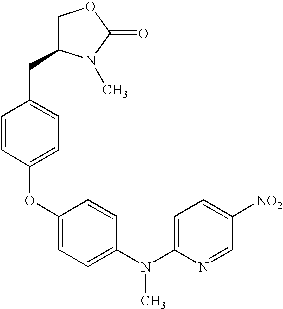 | Yield (0.40 g); ¹HNMR. [CDCl₃, 400 MHz] δ 2.76 (q, 1 H), 2.92 (s, 3 H), 3.11 (dd, 1 H), 3.56 (s, 3 H), 3.93 (m, 1 H), 4.01 (m, 1 H), 4.24 (t, 1 H), 6.34 (d, 1 H), 7.06 (m, 4 H), 7.21 (m, 4 H), 8.04 (dd, 1 H), 9.12 (s, 1 H), m/z^{M+1} 435.46 |
| 34. | 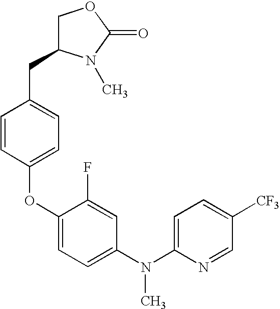 | Yield (0.12 g); ¹HNMR. [CDCl₃, 400 MHz] δ 2.73 (q, 1 H), 2.91 (s, 3 H), 3.11 (dd, 1 H), 3.50 (s, 3 H), 3.93 (m, 1 H), 4.00 (m, 1 H), 4.22 (t, 1 H), 6.55 (d, 1 H), 7.01 (m, 3 H), 7.12 (m, 4 H), 7.53 (dd, 1 H), 8.46 (s, 1 H), m/z^{M+1} 476.1 |
| 35. | 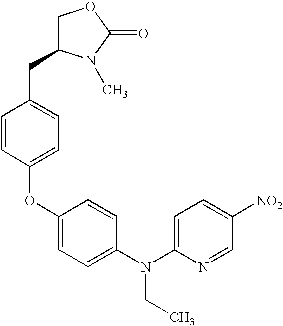 | Yield (0.40 g); ¹HNMR. [CDCl₃, 400 MHz] δ 1.26 (t, 3 H), 2.78 (q, 1 H), 2.92 (s, 3 H), 3.08 (dd, 1 H), 3.93 (m, 1 H), 4.03 (m, 3 H), 4.24 (t, 1 H), 6.20 (d, 1 H), 7.07 (m, 4 H), 7.19 (d, 4 H), 8.01 (dd, 1 H), 9.10 (s, 1 H); m/z^{M+1} 449.1 |
| 36. | 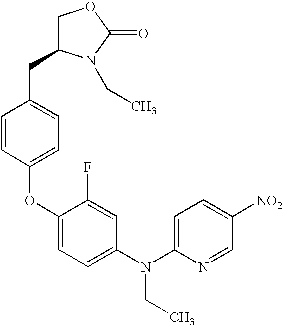 | Yield (0.22); ¹HNMR. [CDCl₃, 400 MHz] δ 1.20 (t, 3 H), 1.27 (t, 3 H), 2.72 (q, 1 H), 3.16 (m, 2 H), 3.61 (m, 1 H), 4.04 (m, 4 H), 4.20 (t, 1 H), 6.27 (d, 1 H), 7.01 (m, 3 H), 7.12 (m, 2 H), 7.16 (d, 2 H), 8.08 (dd, 1 H), 9.11 (s, 1 H); m/z^{M+1} 481.3 |
| 37. | 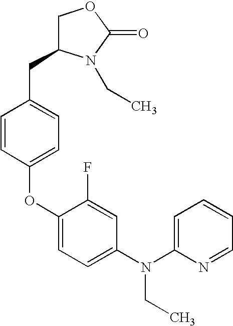 | Yield (0.10); ¹HNMR. [DMSO-d₆, 400 MHz] δ 1.10 (t, 3 H), 1.15 (t, 3 H), 2.72 (m, 1 H), 3.02 (dd, 1 H), 3.13 (m, 1 H), 3.34 (s, 1 H), 3.94 (m, 4 H), 4.14 (t, 1 H), 6.52 (d, 1 H), 6.69 (t, 1 H), 6.98 (d, 2 H), 7.11 (d, 1 H), 7.17 (m, 1 H), 7.33 (m, 3 H), 8.13 (t, 1 H), 8.15 (d, 1 H); m/z^{M+1} 436.3 |
| 38. | 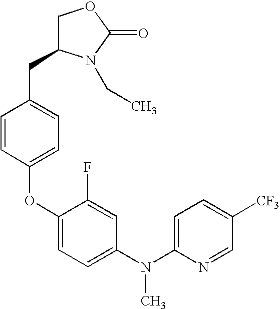 | Yield (0.35 g); ¹HNMR. [CDCl₃, 400 MHz] δ 1.20 (t, 3 H), 2.69 (q, 1 H), 3.10 dd, 1 H), 3.17 (m, 1 H), 3.50 (s, 3 H), 3.60 (m, 1 H), 4.02 (m, 1 H), 4.19 (t, 1 H), 6.55 (d, 1 H), 7.00 (m, 3 H), 7.11 (m, 4 H), 7.61 (dd, 1 H), 8.47 (s, 1 H) m/z^{M+1} 490.3 |
| 39. | 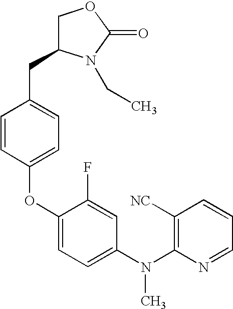 | Yield (0.30 g); ¹HNMR. [CDCl₃, 400 MHz] δ 1.19 (t, 3 H), 2.63 (q, 1 H), 3.11 (m, 2 H), 3.52 (s, 3 H), 3.58 (m, 1 H), 3.98 (q, 2 H), 4.15 (t, 1 H), 6.80 (d, 1 H), 7.04 (m, 3 H), 7.10 (m, 4 H), 7.70 (dd, 1 H), 8.43 (d, 1 H); m/z^{M+1} 447.2 |
| 40. | 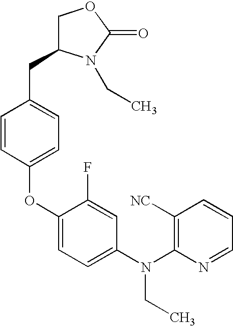 | Yield (0.28 g); ¹HNMR. [CDCl₃, 400 MHz] δ 1.19 (t, 3 H), 1.26 (t, 3 H), 2.60 (m, 1 H), 3.10 (m, 2 H), 3.68 (m, 1 H), 4.01 (m, 4 H), 4.17 (t, 1 H), 6.75 (m, 1 H), 7.02 (m, 3 H), 7.11 (m, 4 H), 7.66 (t, 1 H), 8.40 (d, 1 H); m/z^{M+1} 461.3 |
| 41. | 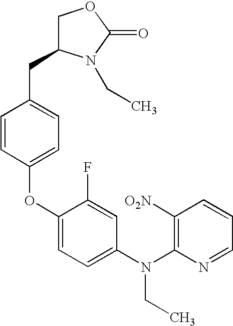 | Yield (0.40 g); ¹HNMR. [CDCl₃, 400 MHz] δ 1.19 (t, 3 H), 1.28 (t, 3 H), 2.60 (m, 1 H), 3.16 (m, 2 H), 3.60 (m, 1 H), 4.00 (q, 2 H), 4.17 (m, 3 H), 6.60 (d, 1 H), 6.91 (m, 4 H), 6.94 (m, 1 H), 7.12 (d, 2 H), 7.96 (dd, 1 H), 8.46 (d, 1 H); m/z^{M+1} 481.2 |
| 42. | 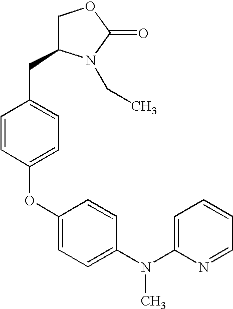 | Yield (0.25 g); ¹HNMR. [CDCl₃, 400 MHz] δ 1.20 (t, 3 H), 2.68 (q, 1 H), 3.13 (m, 2 H), 3.45 (s, 3 H), 3.60 (m, 1 H), 4.03 (q, 2 H), 4.19 (t, 1 H), 6.49 (d, 1 H), 6.60 (m, 1 H), 7.01 (m, 4 H), 7.14 (d, 2 H), 7.24 (m, 2 H), 7.32 (m. 1 H), 8.21 (d, 1 H); m/z^{M+1} 404.1 |
| 43. | 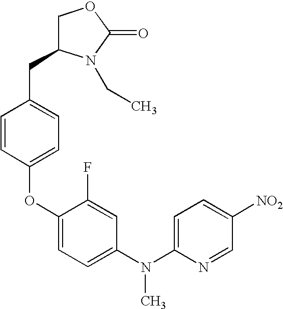 | Yield (0.19 g); ¹HNMR. [CDCl₃, 400 MHz] δ 1.20 (t, 3 H), 2.68 (q, 1 H), 3.16 (m, 2 H), 3.57 (s, 3 H), 3.62 (m, 1 H), 4.01 (m, 2 H), 4.20 (t, 1 H), 6.44 (d, 1 H), 7.03 (m, 3 H), 7.13 (m, 4 H), 8.10 (dd, 1 H), 9.12 (d, 1 H); m/z^{M+1} 467.2 |
| 44. | 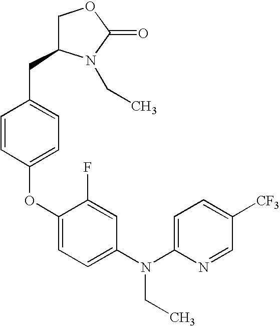 | Yield (0.20 g); ¹HNMR. [CDCl₃, 400 MHz] δ 1.20 (t, 3 H), 1.26 (t, 3 H), 2.68 (q, 1 H), 3.16 (m, 2 H), 3.60 (m, 1 H), 4.00 (q, 4 H), 4.17 (m, 1 H), 6.38 (dd, 1 H), 6.98 (d, 3 H), 7.11 (m, 4 H), 7.49 (dd, 1 H), 8.44 (d, 1 H); m/z^{M+1} 504.1 |
| 45. | 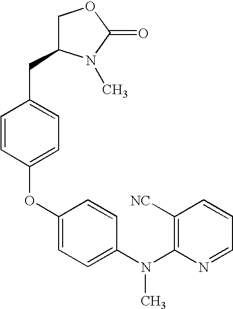 | Yield (0.40 g); ¹HNMR. [CDCl₃, 400 MHz] δ 2.68 (q, 1 H), 2.90 (s, 3 H), 3.09 (dd, 1 H), 3.50 (s, 3 H), 3.89 (t, 1 H), 3.99 (t, 1 H), 4.21 (t, 1 H), 6.72 (q, 1 H), 7.07 (m, 4 H), 7.12 (m, 2 H), 7.23 (m, 2 H), 7.66 (dd, 1 H), 8.40 (d, 1 H); m/z^{M+1} 415.2 |
| 46. | 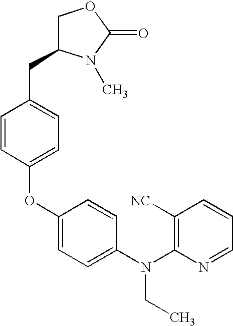 | Yield (0.38 g); ¹HNMR. [CDCl₃, 400 MHz] δ 1.23 (t, 3 H), 2.69 (m, 1 H), 2,90 (s, 3 H), 3.1 (dd, 1 H), 3.89 (m, 1 H), 3.99 (m, 3 H), 4.21 (t, 1 H), 6.67 (m, 1 H), 7.1 (m, 6 H), 7.24 (m, 2 H), 7.62 (dd, 1 H), 8.37 (d, 1 H); m/z^{M+1} 429.3 |
| 47. | 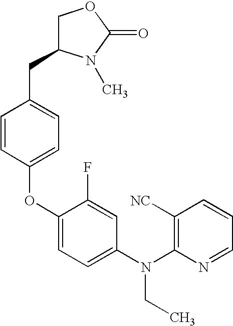 | Yield (0.15 g); ¹HNMR. [CDCl₃, 400 MHz] δ 1.25 (t, 3 H), 2.66 (q, 1 H), 2.89 (s, 3 H), 3.09 (dd, 1 H), 3.88 (m, 1 H), 4.03 (m, 3 H), 4.20 (t, 1 H), 6.74 (q, 1 H), 7.01 (m, 3 H), 7.13 (m, 4 H), 7.67 (d, 1 H), 8.40 (d, 1 H); m/z^{M+1} 447.2 |
| 48. | 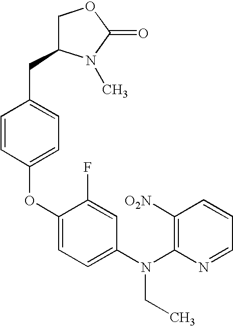 | Yield (0.1 g); ¹HNMR. [CDCl₃, 400 MHz] δ 1.28 (t, 3 H), 2.67 (q, 1 H), 2.89 (s, 3 H), 3.11 (dd, 1 H), 3.89 (m, 1 H), 3.99 (t, 1 H), 4.17 (m, 3 H), 6.76 (d, 1 H), 6.94 (m, 5 H), 7.11 (d, 2 H), 7.96 (d, 1 H), 8.45 (d, 1 H); m/z^{M+1} 467.4 |
| 49. | 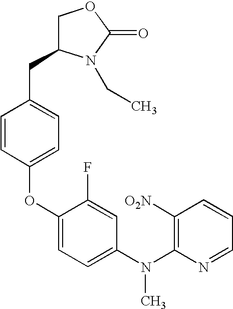 | Yield (0.45 g); ¹HNMR. [CDCl₃, 400 MHz] δ 1.19 (t, 3 H), 2.66 (q, 1 H), 3.16 (m, 2 H), 3.60 (m, 4 H), 4.00 (d, 2 H), 4.17 (t, 1 H), 6.79 (d, 1 H), 6.95 (m, 5 H), 7.11 (d, 2 H), 8.01 (dd, 1 H), 8.49 (d, 1 H); m/z^{M+1} 467.4 |
| 50. | 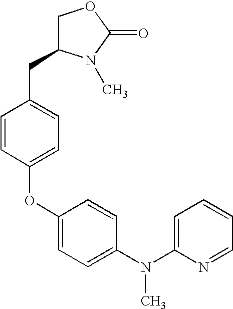 | Yield (0.09 g); ¹HNMR. [CDCl₃, 400 MHz] δ 2.72 (q, 1 H), 2.91 (s, 3 H), 3.09 (dd, 1 H), 3.46 (s, 3 H) 3.92 (m, 1 H), 4.01 (q, 1 H), 4.22 (t, 1 H), 6.49 (d, 1 H), 6.61 (m, 1 H), 7.02 (m, 4 H), 7.14 (m, 2 H), 7.24 (m, 2 H), 7.32 (m, 1 H), 8.22 (d, 1 H); m/z^{M+1} 389.9 |
| 51. | 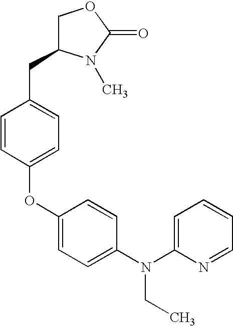 | Yield (0.15 g); ¹HNMR. [CDCl₃, 400 MHz] δ 1.22 (t, 3 H), 2.73 (q, 1 H), 2.91 (s, 3 H), 3.12 (dd, 1 H), 3.97 (m, 4 H), 4.23 (t, 1 H), 6.33 (d, 1 H), 6.56 (m, 1 H), 7.02 (m, 4 H), 7.21 (m, 4 H), 7.28 (m, 1 H), 8.18 (d, 1 H); m/z^{M+1} 404.1 |

### Protocols for Biological Testing

### Glucose Uptake Assay Using 3T3-L1 Cells

3T3-L1 cells were differentiated by the addition of differentiation cocktail (72 μg/ml insulin, 0.5 mM IBMX, 400 ng/ml Dexamethasone) for 4 days and later fed with media without differentiation cocktail for 7-8 days. After differentiation the cells were incubated with the either the reference compound BLX-1002 or compounds listed in the table 1 at 1 μM concentrations for 72 hours and carried out the glucose uptake assay for 10 minutes by the addition of KRP buffer supplemented with 2.5 μCi/ml ¹⁴C deoxy glucose. Stimulation Index is defined as the amount of ¹⁴C Deoxyglucose uptake induced by 1 μM of BLX-1002 incubated for 72 hours in an assay condition as per protocol described above with differentiated 3T3-L1 adipocytes. Values of compounds mentioned in table-1 are with reference to stimulation index of reference compound BLX-1002.

TABLE 1
Effect of compounds on glucose uptake assay in 3T3-L1 cells

| Example No | Stimulation Index |
|---|---|
| 1 | 0.827 |
| 2 | 0.935 |
| 8 | 1.049 |
| 12 | 0.935 |
| 13 | 0.874 |
| 14 | 0.827 |

### Antidiabetic Activity in Streptozotocin Induced Diabetic Mice

Male Swiss albino mice were used in the study at the age of 10 weeks. Diabetes was induced in animals by injecting Streptozotocin by i.p. route at a dose of 200-mg/kg-body weight. 48 hours after Streptozotocin administration, the animals were kept for fasting for 6 hours and blood was collected and plasma separated and glucose was estimated. Animals showing greater than 200 mg/dl glucose levels were considered as diabetic and these animals were randomly distributed into various groups. The compounds listed in the table 2 were administered at a dose of 50-mg/kg body weight by oral route for 7 days. Later animals were fasted for 6 hours and blood was collected and plasma separated. Biochemical estimations like glucose, cholesterol and triglycerides were carried out using the plasma. The effect of compounds mentioned in the table was expressed in terms of percentage reduction in biochemical values as compared to control group. The results are as shown in the table 2.

TABLE 2
Effect of compounds in Streptozotocin
induced diabetic mice model

| Example | % Reduction | | |
|---|---|---|---|
| No | Glucose | Cholesterol | Triglyceride |
| 22 | NR | NR | 5.2 |
| 24 | NR | 15.9 | 13.7 |
| 45 | 39 | 10 | 53.8 |

NR—No Reduction

1. A pyridine derivative of the general formula (I), 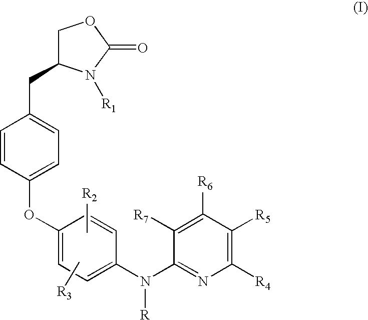
or a stereoisomer or pharmaceutically acceptable salt or pharmaceutically acceptable solvate thereof, wherein, R and R₁ may be same or different and independently represent hydrogen, alkyl, alkenyl, substituted or unsubstituted groups including at least one of (C₁-C₄) alkyl groups; substituted or unsubstituted linear or branched (C₂-C₇) alkenyl groups; substituted or unsubstituted aryl groups; aryloxy groups, substituted or unsubstituted linear or branched (C₂-C₂₀) alkoxy groups; substituted or unsubstituted heteroaryl groups; substituted or unsubstituted heterocyclyl groups; COR₈, where R₈ represents substituted or unsubstituted groups including at least one of (C₁-C₄) alkyl groups; substituted or unsubstituted linear or branched (C₂-C₇) alkenyl groups; substituted or unsubstituted aryl groups; aryloxy groups, or substituted or unsubstituted linear or branched (C₂-C₂₀) alkoxy groups; R₂ and R₃ may be same or different and independently represent hydrogen, halogen; hydroxy, nitro, cyano, formyl, amino, unsubstituted linear or branched (C₁-C₄) alkyl groups; substituted or unsubstituted haloalkyl groups; or substituted or unsubstituted alkoxy groups;
R₄, R₅, R₆ and R₇ may be same or different and independently represent hydrogen, halogen; hydroxy, nitro, cyano, formyl, amino, azido, hydrazine; substituted or unsubstituted groups including at least one of linear or branched (C₁-C₄) alkyl groups; substituted or unsubstituted haloalkyl groups; substituted or unsubstituted alkoxy groups; substituted or unsubstituted monoalkylamino groups; substituted or unsubstituted dialkylamino groups; carboxylic acids, carboxylic acid derivatives; substituted or unsubstituted acylamino groups; substituted or unsubstituted alkylsulfonyl groups; substituted or unsubstituted arylsulfonyl groups; substituted or unsubstituted alkylsulfinyl groups; substituted or unsubstituted arylsulfinyl groups; substituted or unsubstituted alkylthio groups; and alkoxycarbonyl groups.

2. (canceled)

3. (canceled)

4. The compound as claimed in claim 1, wherein the pharmaceutically acceptable salt includes at least one of hydrochloride, hydrobromide, sodium, potassium or magnesium.

5. A pharmaceutical composition comprising a pharmaceutically effective amount of at least one pyridine derivative of formula (I),
as defined in claim 1 or a stereoisomer or pharmaceutically acceptable salt or pharmaceutically acceptable solvate thereof, and a pharmaceutically acceptable carrier, diluent, or excipient.

6. A pharmaceutical composition as claimed in claim 5, in the form of a tablet, capsule, powder, syrup, solution, aerosol or suspension.

7. A pharmaceutical composition as claimed in claim 5, wherein the amount of the at least one pyridine derivative, or a stereoisomer or pharmaceutically acceptable salt or pharmaceutically acceptable solvate thereof, in the composition is less than 60% by weight.

8. A method of reducing at least one of blood glucose, free fatty acids, cholesterol, or triglycerides levels in plasma comprising administering a pharmaceutically effective amount of a at least one pyridine derivative of formula (I) as defined in claim 1 or a stereoisomer or pharmaceutically acceptable salt or pharmaceutically acceptable solvate thereof, to a patient in need thereof.

9. A method of treating at least one of obesity, autoimmune diseases, inflammation, immunological diseases, or cancer comprising administering a pharmaceutically effective amount of at least one pyridine derivative of formula (I) as defined in claim 1 or a stereoisomer or pharmaceutically acceptable salt or pharmaceutically acceptable solvate thereof, to a patient in need thereof.

10. A method of treating a disorder associated with insulin resistance comprising administering a pharmaceutically effective amount of at least one pyridine derivative of formula (I) as defined in claim 1 or a stereoisomer or pharmaceutically acceptable salt or pharmaceutically acceptable solvate thereof, to a patient in need thereof.

11. A method of reducing blood glucose levels in plasma without adipogenic potential comprising administering a pharmaceutically effective amount of at least one pyridine derivative as claimed in claim 1 or a stereoisomer or pharmaceutically acceptable salt or pharmaceutically acceptable solvate thereof to a mammal in need thereof.

12. A method of reducing at least one of TNF-α, IL-6 or IL-β comprising administering a pharmaceutically effective amount of at least one pyridine derivative as claimed in claim 1 or a stereoisomer or pharmaceutically acceptable salt or pharmaceutically acceptable solvate thereof to a mammal in need thereof.

13. A method of reducing cancer cell progression comprising administering a pharmaceutically effective amount of at least one pyridine derivative as claimed in claim 1 or a stereoisomer or pharmaceutically acceptable salt or pharmaceutically acceptable solvate thereof to a mammal in need thereof.

14. A method of reducing blood glucose levels in plasma without adipogenic potential comprising administering a pharmaceutically effective amount of at least one pyridine derivative as claimed in claim 20 or a stereoisomer or pharmaceutically acceptable salt or pharmaceutically acceptable solvate thereof to a mammal in need thereof.

15. A method of reducing at least one of TNF-α, IL-6 or IL-β comprising administering a pharmaceutically effective amount of at least one pyridine derivative as claimed in claim 20 or a stereoisomer or pharmaceutically acceptable salt or pharmaceutically acceptable solvate thereof to a mammal in need thereof.

16. A method of reducing cancer cell progression comprising administering a pharmaceutically effective amount of at least one pyridine derivative as claimed in claim 20 or a stereoisomer or pharmaceutically acceptable salt or pharmaceutically acceptable solvate thereof to a mammal in need thereof.

17. A method of reducing blood glucose levels in plasma without adipogenic potential comprising administering a pharmaceutically effective amount of at least one pyridine derivative as claimed in claim 21 or a stereoisomer or pharmaceutically acceptable salt or pharmaceutically acceptable solvate thereof to a mammal in need thereof.

18. A method of reducing at least one of TNF-α, IL-6 or IL-β comprising administering a pharmaceutically effective amount of at least one pyridine derivative as claimed in claim 21 or a stereoisomer or pharmaceutically acceptable salt or pharmaceutically acceptable solvate thereof to a mammal in need thereof.

19. A method of reducing cancer cell progression comprising administering a pharmaceutically effective amount of at least one pyridine derivative as claimed in claim 21 or a stereoisomer or pharmaceutically acceptable salt or pharmaceutically acceptable solvate thereof to a mammal in need thereof.

20. The pyridine derivative of claim 1, comprising at least one of:
1) (4S)-3-methyl-4-{4-[4-(pyridin-2-ylamino)phenoxy]benzyl}-1,3-oxazolidin-2-one;
2) (4S)4-(4-{4-[(5-nitropyridin-2-yl)amino]phenoxy}benzyl)-3-methyl-1,3-oxazolidin-2-one;
3) (4S)3-methyl-4-[4-(4-{[5-(trifluoromethyl)pyridin-2-yl]amino}phenoxy)benzyl]-1,3-oxazolidin-2-one;
4) (4S)-3-ethyl-4-{4-[4-(pyridin-2-ylamino)phenoxy]benzyl}-1,3-oxazolidin-2-one;
5) (4S)3-ethyl-4-[4-(4-{[5-(trifluoromethyl)pyridin-2-yl]amino}phenoxy)benzyl]-1,3-oxazolidin-2-one;
6) (4S)-4-[4-(2-fluoro-4-{[5-nitropyridin-2-yl]amino}phenoxy)benzyl]-3-ethyl-1,3-oxazolidin-2-one;
7) (4S)-4-[4-(2-fluoro-4-{[5-(trifluoromethyl)pyridin-2-yl]amino}phenoxy)benzyl]-3-ethyl-1,3-oxazolidin-2-one;
8) (4S)-4-[4-(2-fluoro-4-{[5-pyridin-2-yl]amino}phenoxy)benzyl]-3-ethyl-1,3-oxazolidin-2-one;
9) (4S)-4-[4-(2-fluoro-4-{[5-pyridin-2-yl]amino}phenoxy)benzyl]-3-methyl-1,3-oxazolidin-2-one;
10) (4S)-4-[4-(4-{[5-nitropyridin-2-yl]amino}phenoxy)benzyl]-3-ethyl-1,3-oxazolidin-2-one;
11) (4S)-4-[4-(2-fluoro-4-{[5-nitropyridin-2-yl]amino}phenoxy)benzyl]-3-methyl-1,3-oxazolidin-2-one;
12) (4S)-4-[4-(2-fluoro-4-{[3-nitropyridin-2-yl]amino}phenoxy)benzyl]-3-ethyl-1,3-oxazolidin-2-one;
13) 2-{[3-fluoro-4-(4-{[(4S)-3-ethyl-2-oxo-1,3-oxazolidin-4-yl]methyl}phenoxy)phenyl]amino}nicotinonitrile;
14) 2-[(4-{4-[(3-ethyl-2-oxo-1,3-oxazolidin-4-yl)methyl]phenoxy}phenyl)amino]nicotinonitrile;
15) 2-{[4-(4-{[(4S)-3-methyl-2-oxo-1,3-oxazolidin-4-yl]methyl}phenoxy)phenyl]amino}nicotinonitrile;
16) (4S)-3-methyl-4-{4-[4-(3-nitropyridin-2-ylamino)phenoxy]benzyl}-1,3-oxazolidin-2-one;
17) (4S)-4-[4-(2-fluoro-4-{[3-nitropyridin-2-yl]amino}phenoxy)benzyl]-3-methyl-1,3-oxazolidin-2-one;
18) (4S)-4-[4-(2-fluoro-4-{[5-(trifluoromethyl)pyridin-2-yl]amino}phenoxy)benzyl]-3-methyl-1,3-oxazolidin-2-one;
19) (4S)-3-ethyl-4-{4-[4-(3-nitropyridin-2-ylamino)phenoxy]benzyl}-1,3-oxazolidin-2-one;
20) (4S)-4-(4-{2-fluoro-4-[methyl(5-nitropyridin-2-yl)amino]phenoxy}benzyl)-3-methyl-1,3-oxazolidin-2-one;
21) (4S)-2-[methyl(4-{4-[(3-ethyl-2-oxo-1,3-oxazolidin-4-yl)methyl]phenoxy}phenyl)amino]nicotinonitrile;
22) 2-{[3-fluoro-4-(4-{[(4S)-3-methyl-2-oxo-1,3-oxazolidin-4-yl]methyl}phenoxy)phenyl]amino}nicotinonitrile;
23) (4S)-4-(4-{2-fluoro-4-[methyl(3-nitropyridin-2-yl)amino]phenoxy}benzyl)-3-methyl-1,3-oxazolidin-2-one;
24) (4S)-2-[ethyl(4-{4-[(3-ethyl-2-oxo-1,3-oxazolidin-4-yl)methyl]phenoxy}phenyl)amino]nicotinonitrile;
25) (4S)-4-(4-{4-[methyl(3-nitropyridin-2-yl)amino]phenoxy}benzyl)-3-ethyl-1,3-oxazolidin-2-one;
26) (4S)-4-(4-{4-[ethyl(3-nitropyridin-2-yl)amino]phenoxy}benzyl)-3-ethyl-1,3-oxazolidin-2-one;
27) (4S)-4-(4-{2-fluoro-4-[ethyl(5-nitropyridin-2-yl)amino]phenoxy}benzyl)-3-methyl-1,3-oxazolidin-2-one;
28) (4S)-4-(4-{4-[methyl(5-nitropyridin-2-yl)amino]phenoxy}benzyl)-3-ethyl-1,3-oxazolidin-2-one;
29) (4S)-4-(4-{4-[ethyl(5-nitropyridin-2-yl)amino]phenoxy}benzyl)-3-ethyl-1,3-oxazolidin-2-one;
30) 2-[(3-fluoro-4-{4-[(4S)-3-methyl-2-oxo-1,3-oxazolidin-4-yl)methyl]phenoxy}phenyl)(methyl)amino]nicotinonitrile;
31) (4S)-4-(4-{4-[methyl(3-nitropyridin-2-yl)amino]phenoxy}benzyl)-3-methyl-1,3-oxazolidin-2-one;
32) (4S)-4-(4-{4-[ethyl(3-nitropyridin-2-yl)amino]phenoxy}benzyl)-3-methyl-1,3-oxazolidin-2-one;
33) (4S)-4-(4-{4-[methyl(5-nitropyridin-2-yl)amino]phenoxy}benzyl)-3-methyl-1,3-oxazolidin-2-one;
34) (4S)-4-(4-{2-fluoro-4-[methyl(5-(trifluoromethyl)pyridin-2-yl)amino]phenoxy}benzyl)-3-methyl-1,3-oxazolidin-2-one;
35) (4S)-4-(4-{4-[ethyl(5-nitropyridin-2-yl)amino]phenoxy}benzyl)-3-methyl-1,3-oxazolidin-2-one;
36) (4S)-4-(4-{2-fluoro-4-[ethyl(5-nitropyridin-2-yl)amino]phenoxy}benzyl)-3-ethyl-1,3-oxazolidin-2-one;
37) (4S)-4-(4-{2-fluoro-4-[ethyl(pyridin-2-yl)amino]phenoxy}benzyl)-3-ethyl-1,3-oxazolidin-2-one;
38) (4S)-4-(4-{2-fluoro-4-[methyl(5-(trifluoromethyl)pyridin-2-yl)amino]phenoxy}benzyl)-3-ethyl-1,3-oxazolidin-2-one;
39) 2-[(3-fluoro-4-{4-[(4S)-3-ethyl-2-oxo-1,3-oxazolidin-4-yl)methyl]phenoxy}phenyl)(methyl)amino]nicotinonitrile;
40) 2-[(3-fluoro-4-{4-[(4S)-3-ethyl-2-oxo-1,3-oxazolidin-4-yl)methyl]phenoxy}phenyl)(ethyl)amino]nicotinonitrile;
41) (4S)-4-(4-{2-fluoro-4-[ethyl(3-nitropyridin-2-yl)amino]phenoxy}benzyl)-3-ethyl-1,3-oxazolidin-2-one;
42) (4S)-4-(4-{4-[methyl(pyridin-2-yl)amino]phenoxy}benzyl)-3-ethyl-1,3-oxazolidin-2-one;
43) (4S)-4-(4-{2-fluoro-4-[methyl(5-nitropyridin-2-yl)amino]phenoxy}benzyl)-3-ethyl-1,3-oxazolidin-2-one;
44) (4S)-4-(4-{2-fluoro-4-[ethyl(5-(trifluoromethyl)pyridin-2-yl)amino]phenoxy}benzyl)-3-ethyl-1,3-oxazolidin-2-one;
45) (4S)-2-[methyl(4-{4-[(3-methyl-2-oxo-1,3-oxazolidin-4-yl)methyl]phenoxy}phenyl)amino]nicotinonitrile;
46) (4S)-2-[ethyl(4-{4-[(3-methyl-2-oxo-1,3-oxazolidin-4-yl)methyl]phenoxy}phenyl)amino]nicotinonitrile;
47) 2-[(3-fluoro-4-{4-[(4S)-3-methyl-2-oxo-1,3-oxazolidin-4-yl)methyl]phenoxy}phenyl)(ethyl)amino]nicotinonitrile;
48) (4S)-4-(4-{2-fluoro-4-[ethyl(3-nitropyridin-2-yl)amino]phenoxy}benzyl)-3-methyl-1,3-oxazolidin-2-one;
49) (4S)-4-(4-{2-fluoro-4-[methyl(3-nitropyridin-2-yl)amino]phenoxy}benzyl)-3-ethyl-1,3-oxazolidin-2-one;
50) (4S)-4-(4-{4-[methyl(pyridin-2-yl)amino]phenoxy}benzyl)-3-methyl-1,3-oxazolidin-2-one; or
51) (4S)-4-(4-{2-fluoro-4-[ethyl(pyridin-2-yl)amino]phenoxy}benzyl)-3-methyl-1,3-oxazolidin-2-one;
or a pharmaceutically acceptable salt or solvate thereof.

21. The pyridine derivative of claim 1, wherein:
R and R₁ may be same or different and independently represent hydrogen; alkyl; alkenyl; a substituted or unsubstituted methyl, ethyl, propyl, isopropyl, n-butyl, isobutyl, or t-butyl group; a substituted or unsubstituted ethenyl, propenyl, or butenyl group; a substituted or unsubstituted phenyl or naphthyl group; an aryloxy group; a substituted or unsubstituted linear or branched methoxy, ethoxy, propoxy, n-butoxy, isobutoxy, or t-butoxy group; a substituted or unsubstituted pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl, or benzothiazolyl group; a substituted or unsubstituted pyrrolidinyl, thiazolidinyl, oxazolidinyl, morpholinyl, thiomorpholinyl, piperidinyl, or piperazinyl group; COR₈, where R₈ represents substituted or unsubstituted methyl, ethyl, propyl, isopropyl, n-butyl, isobutyl, or t-butyl groups; substituted or unsubstituted ethenyl, propenyl, or butenyl group; substituted or unsubstituted phenyl, or naphthyl groups; aryloxy; substituted or unsubstituted linear or branched methoxy, ethoxy, propoxy, n-butoxy, isobutoxy, or t-butoxy groups;
R₂ and R₃ may be same or different and independently represent hydrogen; fluorine; chlorine; bromine; iodine; hydroxy; nitro; cyano; formyl; amino; methyl; ethyl; n-propyl; isopropyl; n-butyl; isobutyl; t-butyl; substituted or unsubstituted chloromethyl, chloroethyl, trifluoromethyl, trifluoroethyl, dichloromethyl, dichloroethyl, trichloromethyl, or difluoromethyl groups; substituted or unsubstituted methoxy, ethoxy, n-propoxy, or isopropoxy groups;
R₄, R₅, R₆ and R₇ may be same or different and independently represent hydrogen; fluorine; chlorine; bromine; or iodine; hydroxy; nitro; cyano; formyl; amino; azido; hydrazine; substituted or unsubstituted methyl, ethyl, n-propyl, isopropyl, n-butyl, isobutyl, or t-butyl groups;
substituted or unsubstituted chloromethyl, chloroethyl, trifluoromethyl, trifluoroethyl, dichloromethyl, dichloroethyl, trichloromethyl, or difluoromethyl groups; substituted or unsubstituted methoxy, ethoxy, n-propoxy, or isopropoxy groups; substituted or unsubstituted —NHCH₃, —NHC₂H₅, —NHC₃H₇, or —NHC₆H₁₃ groups; substituted or unsubstituted —N(CH₃)₂, —NCH₃(C₂H₅), or —N(C₂H₅)₂ groups; carboxylic acids, carboxylic acid derivatives; esters; amides; substituted or unsubstituted —NHC(═O)CH₃, —NHC(═O)C₂H₅, —NHC(═O)C₃H₇, or —NHC(═O)C₆H₁₃ groups; substituted or unsubstituted methylsulfonyl, ethylsulfonyl, n-propylsulfonyl, or iso-propylsulfonyl groups; substituted or unsubstituted phenylsulfonyl or naphthylsulfonyl groups; substituted or unsubstituted methylsulfinyl, ethylsulfinyl, n-propylsulfinyl, or iso-propylsulfinyl groups; substituted or unsubstituted phenylsulfinyl or naphthylsulfinyl groups; substituted or unsubstituted methylthio, ethylthio, n-propylthio, or iso-propylthio groups; or methoxycarbonyl, ethoxycarbonyl, n-propoxycarbonyl, or isopropoxycarbonyl groups;
or a stereoisomer or pharmaceutically acceptable salt or pharmaceutically acceptable solvate thereof.
